(19) United States
(12) Patent Application Publication
(10) Pub. No.: US 2009/0048575 A1
(43) Pub. Date: Feb. 19, 2009

(54) TROCAR WITH OBTURATOR HAVING LONGITUDINAL THROUGH HOLES FOR GUIDING WIRES

(75) Inventor: Russell David Waters, Kentsford Cheshire (GB)

Correspondence Address: SMITH-HILL AND BEDELL, P.C., 16100 NW CORNELL ROAD, SUITE 220, BEAVERTON, OR, 97006, US

(73) Assignee: BIOCOMPOSITES LIMITED, Keele, Staffordshire (GB)

(21) Appl. No.: 11/577,109

(22) PCT Filed: Sep. 22, 2005
(86) PCT No.: PCT/GB05/03649

§ 371 Date: May 16, 2007

(30) Foreign Application Priority Data
Oct. 13, 2004 (GB) 0422710.4

(51) Int. Cl. A61B 17/34

(52) U.S. Cl. 604/506; 604/164.13

(57) ABSTRACT

Surgical apparatus includes a trocar and obturator. The trocar has a hollow cylindrical body with a handle at the proximal end thereof. The obturator has a solid cylindrical body with a handle at the proximal end thereof. A central through hole is provided extending through the body and a slot is provided in the circumference of the body, extending parallel to the hole. Guide wires can selectively extend through either the central hole or along the slot.

This invention concerns surgical apparatus, particularly but not exclusively surgical apparatus usable in the percutaneous implantation of a compression screw, and also a method of surgery using such apparatus.

Internal fixation of fractured bones is often accomplished with the use of traditional hardware including pins, plates, screws, rods and wire. The clinical objective of the surgeon is to realign the bone fragments in anatomical apposition and to maintain reduction until biological healing has occurred. This is generally achieved through an open procedure where the fracture site is surgically exposed to enable relatively easy access to the bone fragments. However there is a trend towards the use of a more minimally invasive, percutaneous approach since this reduces pain and trauma to the patient and helps a speedier recovery.

The nature of the fracture and the anatomy of the site dictate to some extent the type of hardware that can be used and whether or not a simple percutaneous approach is possible. For highly comminuted fractures multiple pins, screws and/or wires are usually necessary. For simple fractures, particularly those which are undisplaced, a percutaneous approach becomes more possible. The use of compression screws applied across the fracture site is a popular technique. These devices come in a range of configurations including headed or headless, cannulated or solid, fully threaded or partially threaded and with a thread pitch and/or diameter that can vary from the proximal end to the distal end of the screw. They are used across a fracture site to pull together the two bone fragments and hold the fracture surfaces in compression.

The procedure for inserting the screw accurately within the bone and across the fracture site involves initially forming the skin incision followed by the insertion of a guide wire into the bone under x-ray visualisation. The guide wire defines the track of the screw. When the guide wire is in the correct position as judged by the surgeon, a cannulated drill is driven over the said wire to form a bone tunnel for the subsequent insertion of the screw. If the screw is not of the self-tapping type then a tap or bone tunnel dilator may be used prior to screw insertion to form the screw threaded track. This is often the case if the screw is manufactured from a polymer or polymer composite material which does not have the strength to cut into the bone. An effect of all of the processes of drilling, tapping, dilating, countersinking and screwing in the screw is to transmit rotational force to the bone fragments. If these fragments are not held sufficiently firmly then this torque could result in rotation of the bone fragments relative to adjacent tissue or to one another. This would be an undesirable situation.

According to the present invention there is provided surgical apparatus, the apparatus including a trocar and an obturator, the trocar including a handle and a hollow body, which body is locatable so as to extend into an incision in a patient's body during surgery; the obturator being extendible through the trocar body, and including an axial through guide hole through which a guide wire can extend, and a guide formation spaced from the guide hole, which guide formation can guide a stabilising wire.

The guide formation may extend substantially parallel to the guide hole to guide a stabilising wire substantially parallel to and spaced from the guide wire and the guide hole.

Alternatively the guide formation is not parallel to the guide hole, and the guide formation may extend substantially helically around the obturator.

The obturator is preferably a sliding fit in the hollow body of the trocar, and the obturator is preferably rotatable relative to the trocar.

The distal end, relative to a user of the apparatus, of the obturator is preferably extendible beyond the distal end of the trocar body, when the obturator is fully located in the trocar body. The distal end of the obturator is preferably rounded.

A plurality of formations are preferably provided on the distal end of the trocar body, which formations are engageable in use against a bone in a patient's body. The formations may be in the form of teeth spaced circumferentially around the distal end of the trocar body. The teeth are preferably chamfered so as to reduce in length radially outwardly.

The obturator preferably has a substantially cylindrical part which is extendible through the trocar body, and the guide hole preferably extends axially in the cylindrical part.

The guide formation preferably comprises a slot in the circumference of the cylindrical part.

The obturator preferably also includes a proximal formation of a size which cannot extend into the trocar body.

A hole may be provided in the proximal formation which is aligned with the guide formation. The proximal formation may be in the form of a handle to facilitate rotation of the obturator relative to the trocar.

The trocar body is preferably in the form of an open ended hollow cylinder.

The invention also provides a method of surgery, the method including using an apparatus according to any of the preceding eleven paragraphs, passing a guide wire through the guide hole to engage in a patient's body, and passing a stabilising wire along the guide formation to engage in the person's body.

The guide wire may engage in a bone in the person's body.

A hole may be drilled in the bone using a cannulated drill through which the guide wire guidingly extends. The hole may be tapped using a cannulated device through which the guide wire guidingly extends. The hole may be countersunk using a cannulated device through which the guide wire guidingly extends.

A cannulated screw may be inserted in the hole, with the guide wire guidingly extending through the screw and also a tool to turn the screw.

Embodiments of the present invention will now be described by way of example only and with reference to the accompanying drawings, in which:—

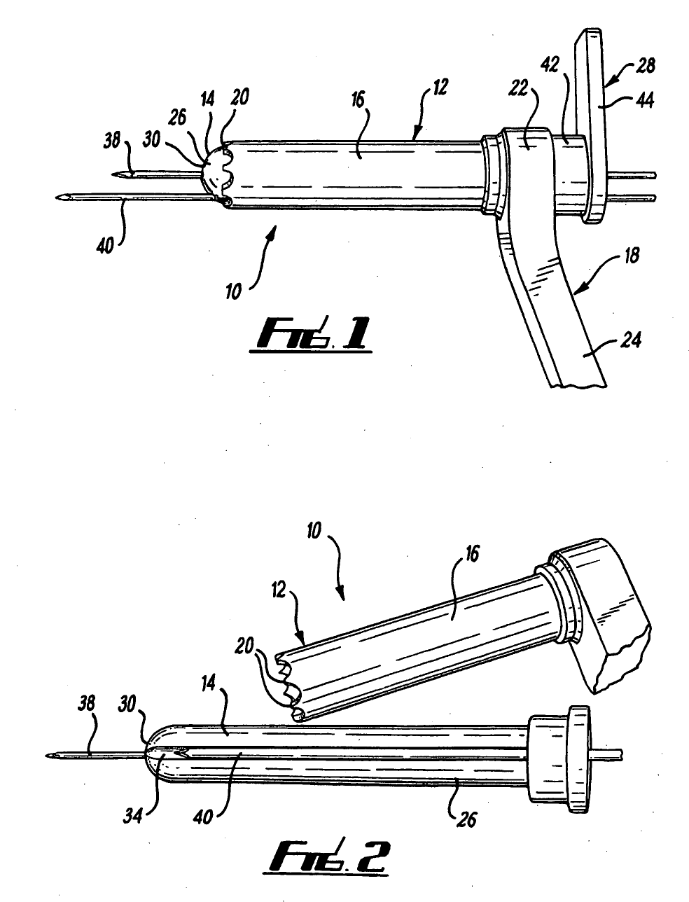

FIG. 1 is a side view of surgical apparatus according to the invention in an assembled condition;

FIG. 2 is a side view of the apparatus of FIG. 1 in a disassembled condition;

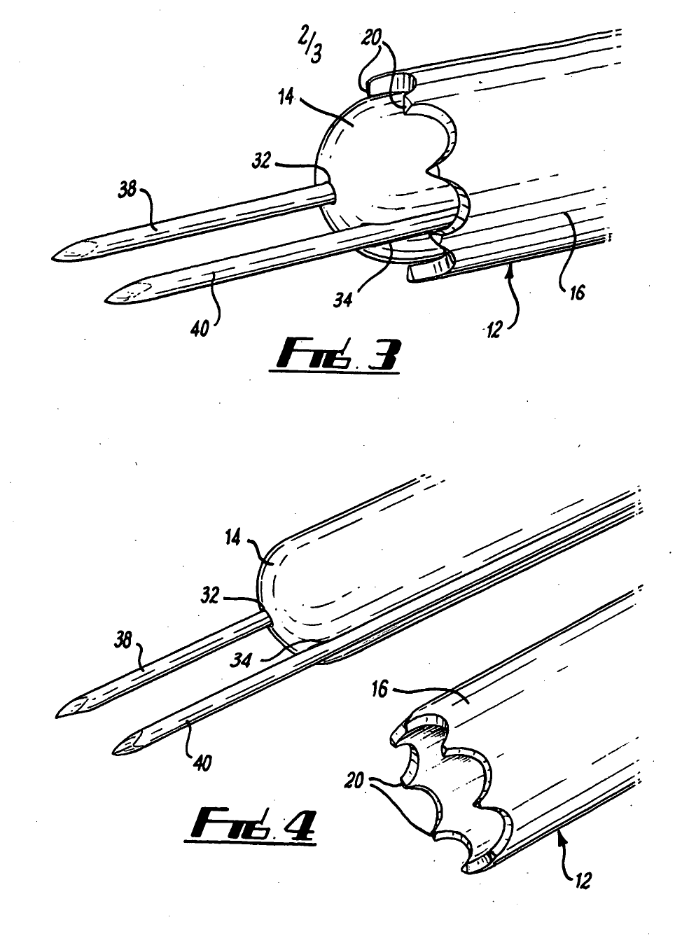

FIG. 3 is a perspective view of the proximal end of the apparatus of FIG. 1 in an assembled condition;

FIG. 4 is a perspective view of the proximal end of the apparatus of FIG. 1 in a disassembled condition;

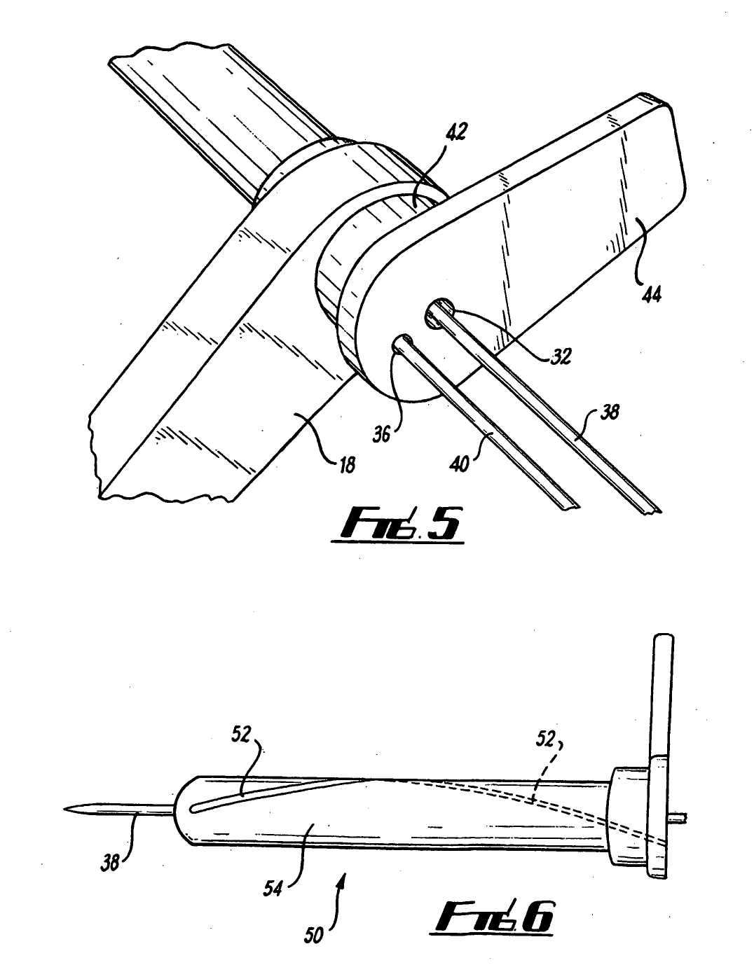

FIG. 5 is a perspective view of the distal end of the apparatus of FIG. 1 in an assembled condition; and

FIG. 6 is a diagrammatic side view of part of an alternative apparatus according to the invention.

FIGS. 1 to 5 of the drawings show surgical apparatus 10 suitable for use in percutaneous bone fixation, for example in implanting a compression screw to join together two bone fragments. The apparatus 10 comprises a trocar 12 and obturator 14. The trocar 12 comprises a hollow cylindrical body 16 with a handle 18 at the proximal end of the body 16.

Engagement formations in the form of a plurality of teeth 20 are provided spaced around the distal end of the body. 16. The teeth 20 are chamfered such that they slope rearwardly away from the axis of the trocar 12. The handle 18 has a through hole corresponding to the interior of the body 16 to enable the obturator 14 to pass therethrough. The handle 18 includes a first section 22 which extends transversely from the proximal end of the body 16, and a second section 24 which extends transversely and also rearwardly.

The obturator 14 comprises a solid cylindrical body 26 with a handle 28 at the proximal end of the body 26. The body 26 has a rounded distal end 30. A central through hole 32 is provided extending through the body 26 and handle 28. A slot 34 is provided in the circumference of the body 26 extending parallel to the through hole 32. A further hole 36 is provided in the handle 28 aligned with the slot 34. The through hole 32 is of a size to slidingly accept a guide wire 38. The slot 34 and further hole 36 are of a size to slidingly accept a stabilising wire 40.

The obturator body 26 is of a size to slidingly and rotatingly fit within the body 16. The obturator handle 28 comprises a first collar section 42 of a size to abut against the trocar handle 18, being too large to pass through the interior of the body 16. The handle 28 also comprises a transverse section 44 at the proximal end of the collar section 42, to facilitate rotation of the obturator 14.

The apparatus 10 can be used for example to perform a surgical procedure to repair a fractured scaphoid. The procedure involves the percutaneous implantation of a cannulated compression screw to hold in apposition the bone fragments and facilitate bony union, and will now be described.

An incision is made over the proximal pole of the scaphoid. The obturator is fully located in the trocar as shown in FIG. 1, and this combination is advanced through the skin and soft tissue, until the teeth 20 on the trocar 12 engage the cortical surface of the first bone fragment. The rounded distal end 30 of the obturator 14 and the chamfered teeth 20 enable percutaneous advancement of the combined trocar 12 and obturator 14 without snagging or tearing of soft tissue. Forward pressure on the trocar 12 maintains fracture reduction while the teeth 20 bite into the cortical bone to prevent slipping of the apparatus 10 on the bone surface. The position on the bone surface adjacent the through hole 32 defines the subsequent position of entry of the compression screw.

The guide wire 38 is then introduced through the hole 32 under x-ray visualisation. The guide wire 38 may be rotated and drilled into the bone fragments. The obturator 14 is then rotated relative to the trocar 12 as required to locate the stabilising wire 40 in a required position. The wire 40 is then introduced through the further hole 36 and along the slot 34 to engage in the two bone fragments. The obturator 14 is removed from the trocar 12 and hence patient. A cannulated drill is passed over the guide wire 38 through the trocar 12 to form a bone tunnel within the bone fragments. In certain circumstances it may be that the trocar 12 is also removed prior to drilling. The rotational motion of the drill conveys a rotational force to the bone fragments which could potentially result in relative misalignment. Such rotation is substantially prevented by the stabilising wire 40.

If the screw to be inserted is a polymeric based device as opposed to a metallic self tapping screw, a tap or bone tunnel dilator will then be inserted over the guide wire 38 to form a track through the screw. If required the hole may also be countersunk. The cannulated compression screw can then be inserted over the guide wire 38 to pull together and hold together the bone fragments in an anatomical position. A cannulated tool to rotate the screw may be inserted over the guide wire 38. The guide wires 38, 40 are then removed prior to wound closure.

There is thus described surgical apparatus and also a method of using such apparatus, which permits percutaneous implantation of a bone compression screw whilst significantly reducing the possibility of misalignment of bone fragments due to rotational forces caused for instance by drilling. The apparatus is of a conventional format and can thus be used in a normal manner without any significant extra surgical steps. The apparatus can also be readily manufactured using conventional techniques.

FIG. 6 shows a further obturator 50 which is similar in most respects to the obturator 14. However, with the obturator 50 a slot 52 is provided in the circumference of the obturator body 54, which slot 52 is not parallel to the central through hole which can receive a guide wire 38. In this instance the slot 52 defines a gentle helix around the body 54. In use a stabilising wire guided by the slot 52 will enter and exit the apparatus inclined relative to the guide wire 38.

Various other modifications may be made without departing from the scope of the invention. For example the shape of the components may be changed. A plurality of stabilising wires could be used, and in different alignments.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-26. (canceled)

27. Surgical apparatus, the apparatus including a trocar and an obturator, the trocar including a handle and a hollow body, which body is beatable so as to extend into an incision in a patient's body during surgery; the obturator being extendible through the trocar body, and including an axial through guide hole through which a guide wire can extend, and a guide formation spaced from the guide hole, which guide formation can guide a stabilizing wire.

28. Apparatus according to claim 27 wherein the guide formation extends substantially parallel to the guide hole to guide a stabilising wire substantially parallel to and spaced from the guide wire and the guide hole.

29. Apparatus according to claim 27 wherein the guide formation is not parallel to the guide hole.

30. Apparatus according to claim 29 wherein the guide formation extends substantially helically around the obturator.

31. Apparatus according to claim 27 wherein the obturator is a sliding fit in the hollow body of the trocar.

32. Apparatus according to claim 27 wherein the obturator is rotatable relative to the trocar.

33. Apparatus according to claim 27 wherein the distal end, relative to a user of the apparatus, of the obturator is extendable beyond the distal end of the trocar body, when the obturator is fully located in the trocar body.

34. Apparatus according to claim 27 wherein the distal end of the obturator is rounded.

35. Apparatus according to claim 27 wherein a plurality of formations are provided on the distal end of the trocar body, which formations are engageable in use against a bone in a patient's body.

36. Apparatus according to claim 35 wherein the formations are in the form of teeth spaced circumferentially around the distal end of the trocar body.

37. Apparatus according to claim 36, wherein the teeth are chamfered so as to reduce in length radially outwardly.

38. Apparatus according to claim 27 wherein the obturator has a substantially cylindrical part which is extendible through the trocar body.

39. Apparatus according to claim 38, wherein the guide hole extends axially in the cylindrical part.

40. Apparatus according to claim 38 wherein the guide formation comprises a slot in the circumference of the cylindrical part.

41. Apparatus according to claim 27 wherein the obturator includes a proximal formation of a size which cannot extend into the trocar body.

42. Apparatus according to claim 41 wherein a hole is provided in the proximal formation which is aligned with the guide formation.

43. Apparatus according to claim 41 wherein the obturator is rotatable relative to the trocar and the proximal formation is in the form of a handle to facilitate rotation of the obturator relative to the trocar.

44. Apparatus according to claim 27 wherein the trocar body is in the form of an open ended hollow cylinder.

45. A method of surgery, the method including using an apparatus according to claim 27 passing a guide wire through the guide hole to engage in a patient's body, and passing a stabilising wire along the guide formation to engage in the person's body.

46. A method according to claim 45 wherein the guide wire engages in a bone in the person's body.

47. A method according to claim 46 wherein a hole is drilled in the bone using a cannulated drill through which the guide wire guidingly extends.

48. A method according to claim 47 wherein the hole is tapped using a cannulated device through which the guide wire guidingly extends.

49. A method according to claim 47 wherein the hole is countersunk using a cannulated device through which the guide wire guidingly extends.

50. A method according to claim 47 wherein a cannulated screw is inserted in the hole, with the guide wire guidingly extending through the screw and also a tool to turn the screw.
